(19) United States
(12) Patent Application Publication
(10) Pub. No.: US 2019/0160298 A1
(43) Pub. Date: May 30, 2019

(54) WIRELESS ELECTROMAGNETIC THERMOTHERAPY APPARATUS

(71) Applicant: METAL INDUSTRIES RESEARCH AND DEVELOPMENT CENTRE, Kaohsiung (TW)

(72) Inventors: Tung-Chieh YANG, Kaohsiung (TW); Yu-Jie LAN, Kaohsiung (TW); Chien-Chang CHEN, Kaohsiung (TW); Szu-Hua YANG, Kaohsiung (TW); Yii-Der WU, Kaohsiung (TW); Tsung-Chih YU, Kaohsiung (TW)

(21) Appl. No.: 15/857,199

(22) Filed: Dec. 28, 2017

(30) Foreign Application Priority Data
Nov. 30, 2017 (TW) 106141786

(51) Int. Cl. A61N 2/02; H02J 50/10; H02J 7/02; H02J 50/80

(52) U.S. Cl. CPC A61N 2/02; H02J 50/80; H02J 7/025; H02J 50/10

(57) ABSTRACT

A wireless electromagnetic thermotherapy apparatus applies a magnetic field on a needle for actuating heat generation thereby, and includes an excitation device and a detector device. The detector device includes a sensor unit that measures a temperature of the needle, a wireless charging unit that generates electrical energy in response to receipt of the magnetic field, a wireless transmitter that is powered by the electrical energy, and that transmits the measured temperature to the excitation device to enable the same to make a comparison between the measured temperature and a target temperature and to adjust an intensity of the magnetic field based on a result of the comparison.

### CROSS-REFERENCE TO RELATED APPLICATION

This application claims priority of Taiwanese Patent Application No. 106141786, filed on Nov. 30, 2017.

### FIELD

The disclosure relates to a thermotherapy apparatus, and more particularly to a wireless electromagnetic thermotherapy apparatus.

### BACKGROUND

Referring to FIG. 1, a conventional electromagnetic thermotherapy apparatus 1 is to be used in combination with an induction needle 11 for treating a body part, such as a tumor or a lesion. The conventional electromagnetic thermotherapy apparatus 1 includes an excitation module 12, two thermocouple wires 13 used to measure temperature and electrically connected with the excitation module 12 through electric cables. In use, the induction needle 11 is disposed on the body part to be treated, and the excitation module 12 is activated to produce an alternating magnetic field that is applied on the induction needle 11 so eddy currents are induced within the induction needle 11. The eddy currents flowing through the resistance of the induction needle 11 dissipate energy as heat, so that the body part can be treated by the heat produced by the induction needle 11. In order to make sure that the induction needle 11 heats up to a target temperature, the two thermocouple wires 13 are disposed adjacent to the induction needle 11 for measuring an actual temperature around the induction needle 11. The actual temperature thus measured is fed back to the excitation module 12 through at least one of the electric cables, and the excitation module 12 is enabled to adjust an intensity of the alternating magnetic field based on the actual temperature for causing the actual temperature to approach the target temperature.

However, since the two thermocouple wires 13 are electrically connected to the excitation module 12 through the electric cables, contributing to creating a messy environment which incurs dangers and inconvenience during the process of treatment.

### SUMMARY

Therefore, an object of the disclosure is to provide a wireless electromagnetic thermotherapy apparatus that can alleviate at least one of the drawbacks of the prior art.

According to the disclosure, the wireless electromagnetic thermotherapy apparatus is configured to apply a magnetic field on a needle which is disposed on a body part to be treated for actuating heat generation by the needle. The wireless electromagnetic thermotherapy device includes an excitation device and a detector device.

The excitation device includes a magnetic field output unit configured to produce the magnetic field for actuating the needle to heat up to a target temperature, and an excitation controller configured to adjust an intensity of the magnetic field produced by the magnetic field output unit.

The detector device is to be coupled to the needle, and includes a sensor unit, a wireless charging unit and a wireless transmitter. The sensor unit is configured to measure an actual temperature of the needle to result in a measured temperature value. The wireless charging unit is configured to generate electrical energy in response to receipt of the magnetic field, and to store the electrical energy thus generated. The wireless transmitter is electrically connected to the sensor unit for obtaining the measured temperature value from the sensor unit, is electrically connected to the wireless charging unit for being powered by the electrical energy thus generated, and is configured to transmit the measured temperature value to the excitation controller of the excitation device through wireless communication, so as to enable the excitation controller to make a comparison between the measured temperature value and a value of the target temperature and to adjust the intensity of the magnetic field, produced by the magnetic field output unit, based on a result of the comparison.

An effect of the disclosure resides in that, by the detector device which is subjected to the magnetic field to produce the electric energy, and which is powered by the electric energy to transmit the measured temperature value measured by the sensor unit to the excitation device through wireless communication, the needle in combination with the wireless electromagnetic thermotherapy apparatus can be operated in a wireless manner so as to promote convenience during a process of treatment.

### BRIEF DESCRIPTION OF THE DRAWINGS

Other features and advantages of the disclosure will become apparent in the following detailed description of the embodiment(s) with reference to the accompanying drawings, of which:

FIG. 1 is a schematic diagram illustrating a conventional electromagnetic thermotherapy apparatus;

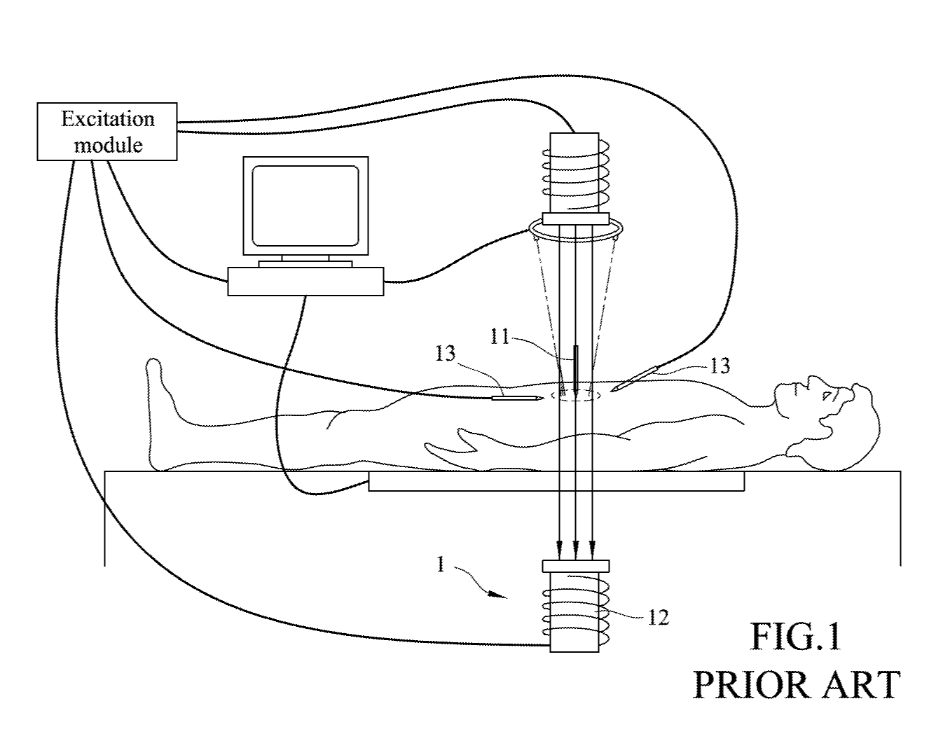

FIG. 2 is a schematic diagram illustrating one embodiment of a wireless electromagnetic thermotherapy apparatus according to the disclosure;

FIG. 3 is a block diagram illustrating one embodiment of electronic components of the wireless electromagnetic thermotherapy apparatus according to the disclosure; and

FIG. 4 is a flow chart illustrating one embodiment of operations to be performed by the excitation controller and the frequency detector of the wireless electromagnetic thermotherapy apparatus.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

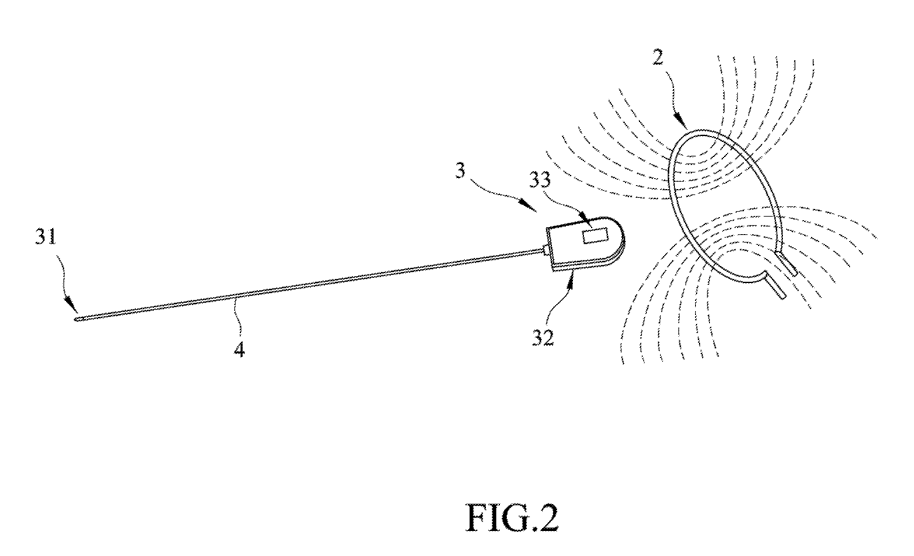

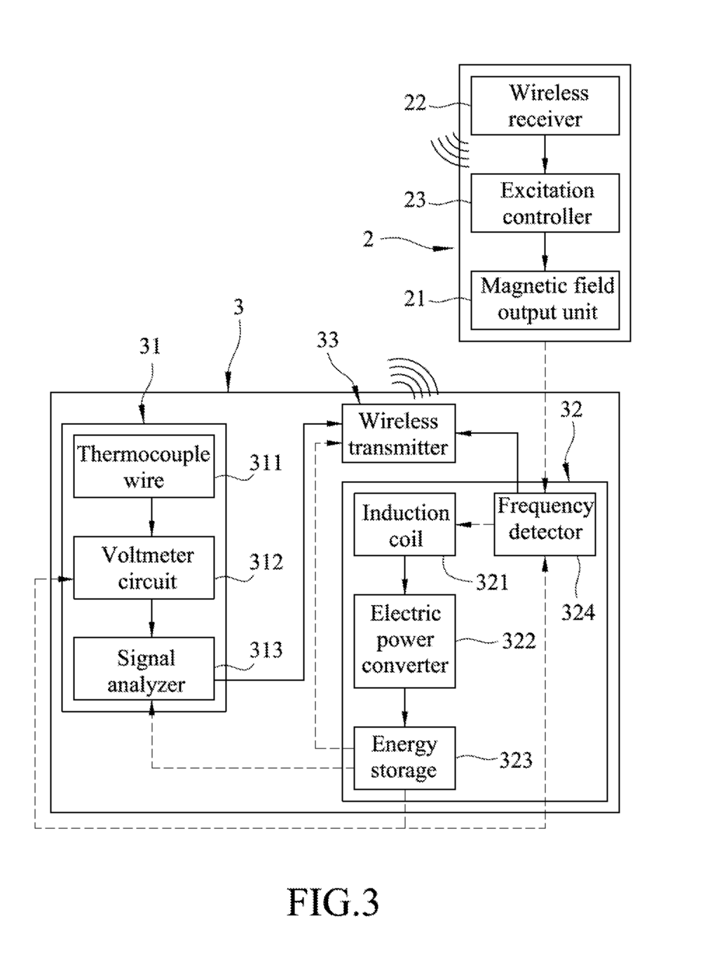

Referring to FIGS. 2 and 3, an embodiment of a wireless electromagnetic thermotherapy apparatus according to this disclosure is configured to apply a magnetic field on a needle 4 which is disposed on a body part to be treated for actuating heat generation by the needle 4. The wireless electromagnetic thermotherapy apparatus includes an excitation device 2 and a detector device 3 which is to be integrated with the needle 4. In other embodiments, the detector device 3 may be combined with the needle 4 in a removable manner.

The excitation device 2 includes a magnetic field output unit 21 configured to produce the magnetic field for actuating the needle 4 to heat up to a target temperature, a wireless receiver 22, and an excitation controller 23 electrically connected between the magnetic field output unit 21 and the wireless receiver 22 and configured to adjust an intensity of the magnetic field produced by the magnetic field output unit 21. The magnetic field output unit 21 is implemented as a coil of wire which is driven by the excitation controller 23 to produce the magnetic field.

The detector device 3 is to be coupled to the needle 4, and includes a sensor unit 31, a wireless charging unit 32, and a wireless transmitter 33 which is electrically connected to the sensor unit 31 and the wireless charging unit 32. The sensor unit 31 is configured to measure an actual temperature of the needle 4 to result in a measured temperature value. The wireless charging unit 32 is configured to generate electrical energy in response to receipt of the magnetic field, and to store the electrical energy thus generated so as to realize inductive charging.

The sensor unit 31 includes a thermocouple wire 311, a voltmeter circuit 312 electrically connected to the thermocouple wire 311, and a signal analyzer 313 electrically connected to the voltmeter circuit 312. The thermocouple wire 311 is to be disposed at a tip of the needle 4, and is configured to produce a voltage according to a temperature around the thermocouple wire 311 (i.e., the actual temperature) as a result of the thermoelectric effect. The voltmeter circuit 312 is configured to measure the voltage produced by the thermocouple wire 311. The signal analyzer 313 receives the voltage measured by and from the voltmeter circuit 312, and is configured to convert the voltage into the measured temperature value. The signal analyzer 313 is implemented as a circuit or a chip programmed to solve an equation representing a relationship between voltage and temperature.

The wireless charging unit 32 includes an induction coil 321, an electric power converter 322 electrically connected to the induction coil 321, an energy storage 323 electrically connected to the electric power converter 322, and a frequency detector 324 electrically connected to the induction coil 321 and the wireless transmitter 33. The induction coil 321 is configured to react with the magnetic field produced by the magnetic field output unit 21, so as to produce the electrical energy through electromagnetic induction. The electric power converter 322 is configured to convert the electrical energy produced by the induction coil 321 from one form to a desired form, such as a direct current at 5 volts, which can be utilized by electronic components in the detector device 3, namely the voltmeter circuit 312, the signal analyzer 313, the wireless transmitter 33 and the frequency detector 324. The energy storage 323 is configured to store the electrical energy in the desired form, and the voltmeter circuit 312, the signal analyzer 313, the wireless transmitter 33 and the frequency detector 324 are powered by the electrical energy stored in the energy storage 323. The electric power converter 322 is implemented as a rectifier, an AC-to-DC converter, a voltage converter, a frequency converter or any combination thereof. In this embodiment, a stand-alone wireless power receiver IC, BD57015GWL, available from ROHM Semiconductor is used for realizing the electric power converter 322. The energy storage 323 is implemented as a rechargeable battery, a capacitor or a combination thereof. However, implementations of the electric power converter 322 and the energy storage 323 are not limited to the disclosure herein.

In this embodiment, the magnetic field is a time-varying magnetic field or an alternating magnetic field. The frequency of the magnetic field produced by the magnetic field output unit 21 is implemented to range between 30 KHz and 100 KHz. The wireless transmitter 33 and the wireless receiver 22 are respectively exemplified as a Bluetooth transmitter and a Bluetooth receiver for realizing short-range wireless communication therebetween, and operate at the frequency of 2.4 GHz to prevent interference with the magnetic field which is utilized for induction heating and inductive charging. However, other technologies, such as Wi-Fi can be adopted for realizing the short-range wireless communication.

When the wireless electromagnetic thermotherapy apparatus is in use, the needle 4 is first disposed on the body part to be treated. Next, the excitation controller 23 is provided with the target temperature, and drives the magnetic field output unit 21 according to the target temperature to produce the magnetic field such that the magnetic field is applied to a position of the body part to be treated. Under influence of the magnetic field, electromotive force is induced in the needle 4 which incurs heat generation by the needle 4. It should be noted that the actual temperature of the needle 4 may be unequal to the target temperature provided to the excitation controller 23. Therefore, the thermocouple wire 311 disposed at the tip of the needle 4 produces the voltage according to the actual temperature of the needle 4, and the voltmeter circuit 312 measures the voltage produced by the thermocouple wire 311. The signal analyzer 313 converts the voltage measured by the voltmeter circuit 312 into the measured temperature value. The measured temperature value is then outputted to the wireless transmitter 33 for being transmitted thereby via the short-range wireless communication to the wireless receiver 22 of the excitation device 2. Finally, the excitation controller 23 receives the measured temperature value via the wireless receiver 22, makes a comparison between the measured temperature value and a value of the target temperature, and adjusts the intensity of the magnetic field, produced by the magnetic field output unit 21, based on a result of the comparison for causing the actual temperature of the needle 4 to approach the target temperature.

Referring to FIGS. 2 to 4, since the frequency of the magnetic field may be altered according to different needs during a process of treatment, the efficiency of inductive charging to be implemented by the wireless charging unit 32 may be adversely influenced. Therefore, in this embodiment, the induction coil 321 is embedded with a frequency tuning circuit which is exemplified by an integrated circuit (IC) in combination with a passive component, such as a variable resistor, a variable capacitor, an adjustable inductor or any combination thereof. The frequency tuning circuit may be controlled for tuning an operating frequency of the induction coil 321. Moreover, the frequency detector 324 of the wireless charging unit 32 is configured to detect the frequency of the magnetic field that is produced by the magnetic field output unit 21 and a frequency of an induced electromagnetic field of the induction coil 321 which is induced by the magnetic field. When it is determined by the frequency detector 324 that the frequency of the induced electromagnetic field of the induction coil 321 is either lower or higher than the frequency of the magnetic field produced by magnetic field output unit 21, the frequency detector 324 transmits a command to the frequency tuning circuit embedded in the induction coil 321 so as to control the frequency tuning circuit to adjust an operating frequency of the induction coil 321 for matching that of the magnetic field produced by the magnetic field output unit 21 so that the induction coil 321 produces a maximum amount of the electrical energy when being subjected to the magnetic field by virtue of resonant inductive coupling. In this embodiment, the frequency detector 324 includes a phase frequency detector (PFD) and an IC that is programmed to achieve the functions of frequency comparison and of controlling the frequency tuning circuit embedded in the induction coil 321 to adjust the operating frequency of the induction coil 321, so as to optimize the efficiency of inductive charging. It should be noted that other reasons why the frequency of the induced electromagnetic field is lower or higher than the frequency of the magnetic field may be that a position of the needle 4 is adjusted, or a patient breathes or subtly moves his/her body.

On the other hand, when the needle 4 is removed from the body part to be treated or the patient immensely rotates his/her body, it is determined by the frequency detector 324 that no induced electromagnetic field is detected from the induction coil 321 due to the fact that an included angle between the needle 4 and the magnetic field is greater than 45 degrees. As a result, the frequency detector 324 provides a stop signal to the wireless transmitter 33 which then transmits the stop signal to the excitation controller 23 via the wireless receiver 22 of the excitation device 2 through the short-range wireless communication for stopping the magnetic field output unit 21 from producing the magnetic field. In this way, waste of energy may be prevented.

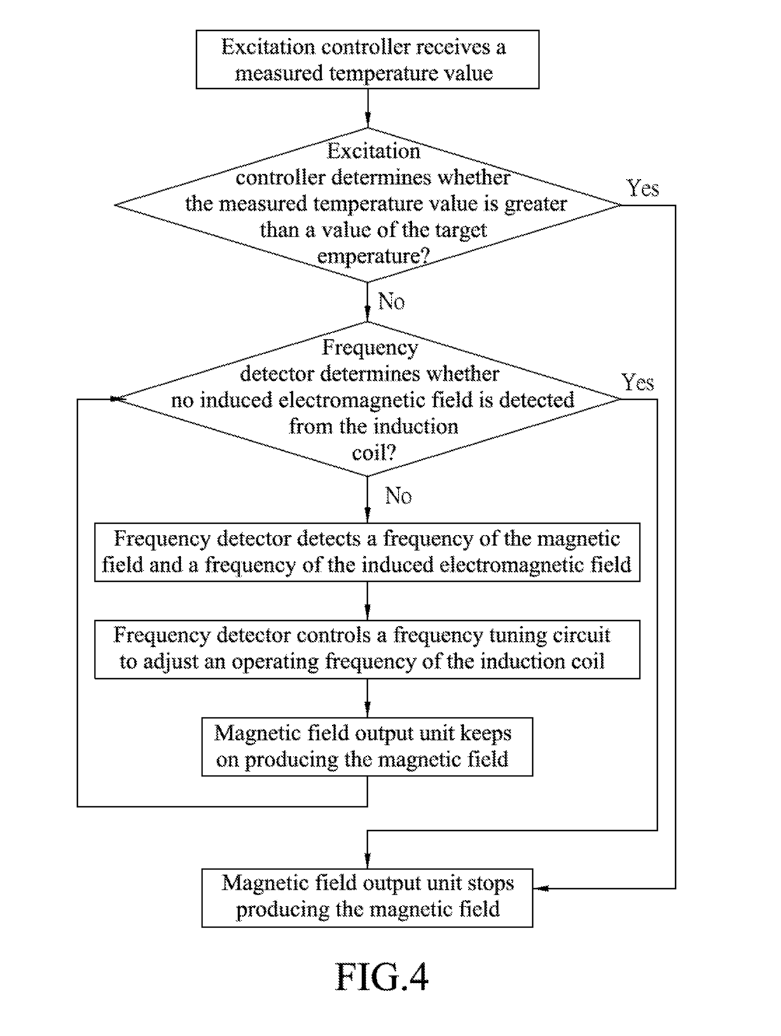

Referring to FIG. 4, in one embodiment, the excitation controller 23 receives the measured temperature value via the wireless receiver 22, and determines whether the measured temperature value is greater than the value of the target temperature. When it is determined that the measured temperature value is not greater than the value of the target temperature, the excitation controller 23 keeps controlling the magnetic field output unit 21 to produce the magnetic field. After that, the frequency detector 324 determines whether no induced electromagnetic field is detected from the induction coil 321. When it is determined that the induced electromagnetic field is detected from the induction coil 321, the frequency detector 324 provides a continue signal to the wireless transmitter 33 which then transmits the continue signal to the excitation controller 23 via the wireless receiver 22 of the excitation device 2 through the short-range wireless communication for controlling the magnetic field output unit 21 to keep on producing the magnetic field. It should be noted that the frequency detection, frequency comparison and control of the frequency tuning circuit are to be performed by the frequency detector 324 only when it is determined by the excitation controller 23 that the measured temperature value is not greater than the value of the target temperature.

In summary, by virtue of the wireless charging unit 32, aside from inducing the electromotive force in the needle 4 to generate heat, the magnetic field produced by the magnetic field output unit 21 can also be received by the induction coil 321 for generating electrical energy through electromagnetic induction where the generated electrical energy is utilized to power the other electronic components. In this way, the wireless electromagnetic thermotherapy apparatus may make full use of the magnetic field. Moreover, if the actual temperature of the needle 4 deviates from the target temperature, the magnetic field output unit 21 is controlled to adjust the magnetic field outputted thereby so the actual temperature can approach the target temperature. In addition, since the measured temperature value is transmitted by the wireless transmitter 33 through wireless communication, bothersome cords or cables for signal transmission may be omitted, so as to ensure a safe and tidy environment for treatment.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A wireless electromagnetic thermotherapy apparatus configured to apply a magnetic field on a needle which is disposed on a body part to be treated for actuating heat generation by the needle, said wireless electromagnetic thermotherapy device comprising:
an excitation device including a magnetic field output unit configured to produce the magnetic field for actuating the needle to heat up to a target temperature, and an excitation controller configured to adjust an intensity of the magnetic field produced by the magnetic field output unit; and
a detector device to be coupled to the needle, and including
a sensor unit configured to measure an actual temperature of the needle to result in a measured temperature value,
a wireless charging unit configured to generate electrical energy in response to receipt of the magnetic field, and to store the electrical energy thus generated, and
a wireless transmitter electrically connected to said sensor unit for obtaining the measured temperature value from said sensor unit, electrically connected to said wireless charging unit for being powered by the electrical energy generated thereby, and configured to transmit the measured temperature value to said excitation controller of said excitation device through wireless communication, so as to enable said excitation controller to make a comparison between the measured temperature value and a value of the target temperature and to adjust the intensity of the magnetic field, produced by said magnetic field output unit, based on a result of the comparison.

2. The wireless electromagnetic thermotherapy apparatus of claim 1, wherein said sensor unit includes:
a thermocouple wire to be disposed adjacent to the needle, and configured to produce a voltage according to a temperature around said thermocouple wire;
a voltmeter circuit electrically connected to said thermocouple wire, and configured to measure the voltage produced by said thermocouple wire; and
a signal analyzer electrically connected to said voltmeter circuit for receiving the voltage measured by said voltmeter circuit, and configured to convert the voltage into the measured temperature value.

3. The wireless electromagnetic thermotherapy apparatus of claim 1, wherein said wireless charging unit includes:
an induction coil configured to react with the magnetic field to produce the electrical energy through electromagnetic induction;
an electric power converter electrically connected to said induction coil, and configured to convert the electrical energy produced by said induction coil from one form to a desired form; and
an energy storage electrical connected to said electric power converter, and configured to store the electrical energy in the desired form.

4. The wireless electromagnetic thermotherapy apparatus of claim 3, wherein said induction coil is embedded with a frequency tuning circuit, and said wireless charging unit further includes:
a frequency detector electrically connected to said induction coil, and configured to
detect a frequency of the magnetic field that is produced by said magnetic field output unit and a frequency of an induced electromagnetic field of said induction coil that is induced from the magnetic field, and
control, based on the frequency of the magnetic field and the frequency of the induced electromagnetic field thus detected, said frequency tuning circuit to adjust an operating frequency of said induction coil for matching that of the magnetic field produced by the magnetic field output unit so that said induction coil produces a maximum amount of the electrical energy when being subjected to the magnetic field.

5. The wireless electromagnetic thermotherapy apparatus of claim 4, wherein said frequency detector is further electrically connected to said wireless transmitter, and is further configured to, when it is determined by said frequency detector that no induced electromagnetic field is detected from said induction coil, provide a stop signal to said wireless transmitter which transmits the stop signal to said excitation controller of said excitation device through the wireless communication for stopping said magnetic field output unit from producing the magnetic field.
